(19) United States
(12) Patent Application Publication
(10) Pub. No.: US 2025/0019633 A1
(43) Pub. Date: Jan. 16, 2025

(54) BIOREACTOR LIQUID AGITATION DEVICE

(71) Applicant: Plant Cell Technology, Inc., Washington, DC (US)

(72) Inventor: John Banner McEverett, Hendersonville, NC (US)

(73) Assignee: Plant Cell Technology, Inc., Washington, DC (US)

(21) Appl. No.: 18/742,706

(22) Filed: Jun. 13, 2024

Related U.S. Application Data
(60) Provisional application No. 63/513,388, filed on Jul. 13, 2023.

(51) Int. Cl. C12M 3/06; C12M 1/00; C12N 5/00

(52) U.S. Cl. CPC C12M 27/14; C12M 21/02; C12N 5/0025

(57) ABSTRACT

A bioreactor liquid agitation device may include a rack which may be movably coupled to a frame. Preferably, a motivator which may be configured to motivate the rack in a movement circuit. A rack may comprise one or more sets of rails. Each rail may comprise a channel which may be formed by two opposing retaining walls which may be coupled to and separated by a central wall. A set of two rails, with each rail having its channel facing the other rail, may be configured to support one or more bioreactors by receiving opposing portions of a vessel coupler of each bioreactor in the two channels. An arrester may be coupled to the rack(s), and the arrestor may block the vessel couplers from exiting an end of the channels so that the vessel couplers may only enter and exit the channels from an opposing end of the channels.

### CROSS REFERENCE TO RELATED APPLICATIONS

This application claims priority to and the benefit of the filing date of U.S. Provisional Application No. 63/513,388, filed on Jul. 13, 2023, entitled “BIOREACTOR LIQUID AGITATION DEVICE”, which is hereby incorporated by reference in its entirety.

### FIELD OF THE INVENTION

This patent specification relates to the field of immersion bioreactor liquid agitation devices. More specifically, this patent specification relates to a device which is configured to receive and agitate a plurality of bioreactor devices, such as which may be used for the aseptic liquid culture of plants and for obtaining exudates from the plants.

### BACKGROUND

Many individuals utilize bioreactors for the aseptic liquid culture of plants, for obtaining exudates from plants, for fermentation under controlled conditions, and for many other purposes. These individuals frequently agitate the bioreactors to facilitate this process and may use wave rocker tables or shelves to provide automatic agitation. However, existing wave rocker tables or shelves are complex, are difficult to operate, and require the use of proprietary containers or vessels which users must typically transfer the propagules from their small-scale bioreactors to the proprietary containers or vessels. Additionally, many existing bioreactors require the utilization of complicated valves, tubing, pumps, and filters which can further hinder the ability to agitate these bioreactors. For this reason, many users choose to manually agitate their bioreactors which can be time consuming and inconvenient.

Therefore, a need exists for novel bioreactor liquid agitation devices. A further need exists for novel agitation devices which may be configured to automatically agitate one or more bioreactors of various makes and models. There is also a need for novel bioreactor liquid agitation devices which are not complex, difficult to operate, or are expensive to produce.

### BRIEF SUMMARY OF THE INVENTION

A bioreactor liquid agitation device is provided which may be used with and/or may comprise one or more bioreactors, each bioreactor having a first vessel and a second vessel which are coupled together via a vessel coupler so that the vessels are in fluid communication with each other. The bioreactor liquid agitation device may be used for both synthetic seed and traditional seed germination, aseptic liquid culture of plants, for obtaining exudates from plants, for fermentation under controlled conditions, and for many other purposes. Being able to germinate synthetic seeds with existing devices is commonly regarded as being impossible, however the use of the bioreactor liquid agitation device of the present invention consistently enables the germination of synthetic seeds.

In some embodiments, the device may include a rack that may be movably coupled to a frame. The rack may have a first rail, a second rail, and an arrestor. The first rail may have a first channel formed by a first upper retaining wall and a first lower retaining wall that are each coupled to a first central wall, and the first channel may have a first distal end and a first proximal end. The second rail may have a second channel formed by a second upper retaining wall and a second lower retaining wall that are each coupled to a second central wall, and the second channel may have a second distal end and a second proximal end. The first channel may face the second channel, and the arrestor may be coupled to the first rail so that the first proximal end forms a portion of the first channel that is closest to the arrestor. A motivator may be configured to move the rack relative to the frame.

In further embodiments, the device may include a rack that may be movably coupled to a frame. The rack may have a first rail, a second rail, and an arrestor. The first rail may have a first channel formed by a first upper retaining wall and a first lower retaining wall that are each coupled to a first central wall, and the first channel may have a first distal end and a first proximal end. The second rail may have a second channel formed by a second upper retaining wall and a second lower retaining wall that are each coupled to a second central wall, and the second channel may have a second distal end and a second proximal end. The first channel may face the second channel, and the arrestor may be coupled to the first rail so that the first proximal end forms a portion of the first channel that is closest to the arrestor. The device may be used with and/or may comprise one or more bioreactors, each bioreactor having a first vessel and a second vessel which are coupled together via a vessel coupler and the vessel coupler providing fluid communication between the first vessel and the second vessel. The bioreactor may be removably coupled to the rack by positioning the vessel coupler in the first channel through the first distal end and in the second channel through the second distal end, and the arrestor may prevent the vessel coupler from exiting the first channel through the first proximal end. A motivator may be configured to move the rack relative to the frame.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a front elevation view of an example of a bioreactor liquid agitation device according to various embodiments described herein.

FIG. 2 illustrates a first side elevation view of an example of a bioreactor liquid agitation device according to various embodiments described herein.

FIG. 3 shows a second side elevation view of an example of a bioreactor liquid agitation device according to various embodiments described herein.

FIG. 4 depicts a third side elevation view of an example of a bioreactor liquid agitation device according to various embodiments described herein.

FIG. 5 illustrates a front elevation view of another example of a bioreactor liquid agitation device according to various embodiments described herein.

FIG. 6 shows a sectional, through line 6-6 shown in FIG. 5, plan view of a portion of an example rack according to various embodiments described herein.

FIG. 7 depicts a front elevation view of a portion of an example rack according to various embodiments described herein.

FIG. 8 illustrates a sectional, through line 8-8 shown in FIG. 5, elevation view of an example bioreactor according to various embodiments described herein

FIG. 9 shows a block diagram of an example of a bioreactor liquid agitation device according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

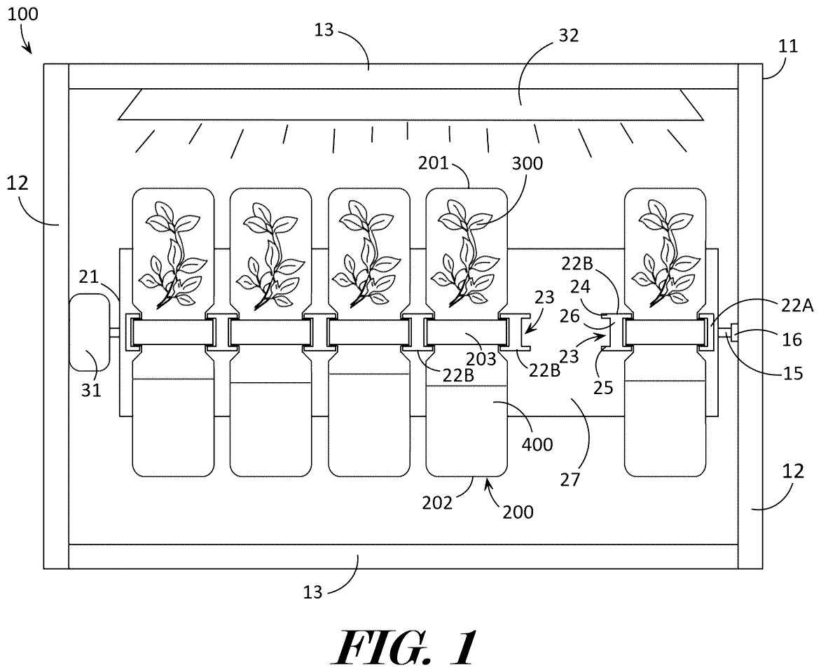

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 15% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

A new bioreactor liquid agitation device is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

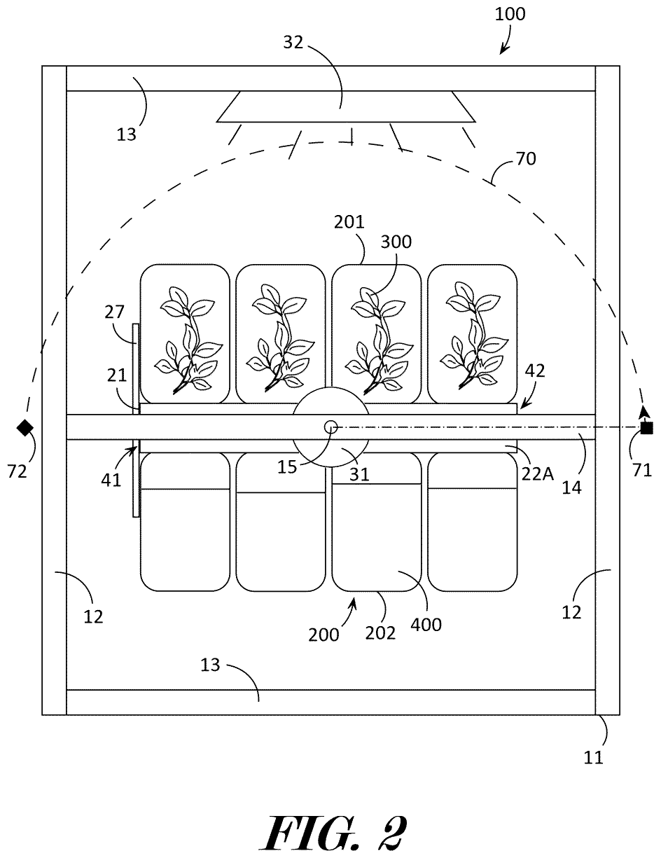

The present invention will now be described by example and through referencing the appended figures representing preferred and alternative embodiments. FIG. 1 illustrates an example of a bioreactor liquid agitation device (“the device”) 100 according to various embodiments. The device 100 may be used with and/or may comprise one or more bioreactors 200, each bioreactor 200 having a first vessel 201 and a second vessel 202 which are coupled together via a vessel coupler 203 so that the vessels 201, 202, are in fluid communication with each other. Generally, bioreactors 200 may be used for the aseptic liquid culture of plants 300 and for obtaining liquid exudates 400 from the plants. In some embodiments, the device 100 may comprise one or more racks 21 which may be movably coupled to a frame 11. Preferably, the device 100 may comprise a motivator 31 which may be configured to motivate the racks 21 in a movement circuit 70 (FIGS. 2-4). Optionally, a rack 21 may comprise one or more rails 22A, 22B. Each rail 22A, 22B, may comprise a channel 23 which may be formed by two opposing retaining walls 24, 25, which may be coupled to and separated by a central wall 26. A set of two rails 22A, 22B, with each rail 22A, 22B, having a channel 23 facing the other rail 22A, 22B, may be configured to support one or more bioreactors 200 by receiving opposing portions of the vessel coupler 203 of each bioreactor 200 into the two channels 23. An arrestor 27 may be coupled to one or more rails 22A, 22B, and the arrestor 27 may block the vessel couplers 203 from exiting a proximal end 41 of the channels 23 so that the vessel couplers 203 may only exit the channels 23 from an opposing distal end 42 of the channels 23.

Optionally, the device 100 may be used with and/or may comprise one or more bioreactors 200. Generally, a bioreactor 200 may comprise an immersion bioreactor device which may be used for, purposes that may include, the aseptic liquid culture of plants, obtaining exudates from plants, fermentation under controlled conditions, and many other purposes. In some embodiments, a bioreactor 200 may include a vessel coupler 203, a first vessel 201, and a second vessel 202. The first vessel 201 may include a first cavity 205 and a first vessel aperture 207 that may be in fluid communication with the first cavity 205. The second vessel 202 may include a second cavity 206 and a second vessel aperture 208 that may be in fluid communication with the second cavity 206. Vessels 201, 202, may comprise containers, such as jars, bottles, etc., that preferably may be made of a transparent or translucent material, such as glass, clear plastic, etc., that may be suitable for containing a volume of liquid and solid material in their cavities 205, 206.

The vessel coupler 203 may be configured to be removably coupled to the first vessel 201 so that the first vessel aperture 207 is in fluid communication with the vessel coupler 203, and the vessel coupler 203 may be also configured to be removably coupled to the second vessel 202 so that the second vessel aperture 208 is also in fluid communication with the vessel coupler 203.

The vessel coupler 203 may comprise a plate 209 which may separate the vessel apertures 207, 208, from each other, and the vessel coupler 203 may comprise a coupling wall 211 that may be coupled to the plate 209 which may be configured to be removably coupled to vessels 201, 202, proximate to their vessel apertures 207, 208. Optionally, vessels 201, 202, may be removably coupled to a coupling wall 211 of a vessel coupler 203 via threading, other turn to lock connection method, or other removable coupling method which may provide a water tight but releasable seal between a vessel 201, 202, and a vessel coupler 203.

One or more communication apertures 204 e.g., holes, cutouts, etc., may be disposed in the plate 209, and the communication apertures 204 may enable fluid communication of liquid exudates 400 between the vessel apertures 207, 208, while preventing plants 300 and plant particulate matter from passing through the vessel coupler 203. In some embodiments, a vessel coupler 203 may comprise a port 210 which may be coupled to an element of a gas supply module 33, such as tubing 36, and the port may enable a gas, such as carbon dioxide, air, etc., to flow into the cavities 205, 206, of the vessels 201, 202, of the bioreactor 200. A port 210 may comprise an aperture that may allow gas to pass through the vessel coupler 203, such as into and through a communication aperture 204 or through the plate 209. Optionally, a port 210 may comprise a check valve to allow gas to enter and/or exit the bioreactor 200 while preventing liquids and liquid exudates 400 from exiting the bioreactor 200.

In some embodiments, the device 100 may comprise a frame 11 which may be configured to support one or more racks 21 above a surface or object that the frame 11 is resting on. Preferably, a rack 21 may be movably coupled to the frame 11 so that the rack 21 may move relative to the frame 11. Optionally, the device 100 may comprise a frame 11 which may be configured to support two or more racks 21 by movably coupling the racks 21 to the frame 11. In still further embodiments, the device 100 may comprise two or more frames 11 which may each be configured to support one or more racks 21 by movably coupling the racks 21 to the respective frames 11.

A frame 11 may be configured in any size and shape which may enable the frame 11 to support one or more racks 21 above or away from a surface or object that the frame 11 may be resting on or coupled to. For example, a frame 11 may comprise a generally rectangular prism shape having a number of vertical supports 12 and a number of horizontal supports 13 which may be coupled together at approximately right angles to form a rectangular prism shape. In further embodiments, the device 100 may comprise one or more vertical supports 12 and and/or horizontal supports 13 that may be curved, angled, or otherwise shaped and sized to form a frame 11 of any shape and size. Preferably, the frame 11 may comprise two rack supports 14 which may be opposingly positioned to each other on the frame 11, and two opposing sides of a rack 21 may be movably coupled to a respective rack support 14 in order to movably couple the rack 21 to the frame 11. Supports 12, 13, 14, may comprise metal, plastic, wood, ceramic, or any other substantially rigid material optionally in the form of tubing, rods, bar stock, plate stock, or any other structural configuration.

In preferred embodiments, the device 100 may comprise one or more transportation conveyances 17 which may be coupled to the frame 11, such as to a vertical support 12, horizontal support 13, etc. For example, the device 100 may comprise four transportation conveyances 17 that may be coupled to opposing sides of the frame 11. The transportation conveyances 17 may support the frame 11 above a ground surface while reducing the friction resistance between the frame 11 and the ground surface during movement of the device 100. A transportation conveyance 17 may comprise a wheel, a caster, a tread or track, a low friction pad or bumper, a low friction plate, or any other suitable device configured to reduce the friction between the device 100 and the surface over which it is desired to be moved. In preferred embodiments, a transportation conveyance 17 may comprise a swivel caster which allows for movement in multiple directions. Typically, swivel casters have one or two sets of raceways that allow the caster to swivel 360 degrees under a load. Swivel casters may include locking casters, kingpin-less casters, hollow kingpin casters, plate casters, stem casters, or any other suitable caster which allows for movement, preferably in multiple directions.

A rack 21 may be movable coupled to a rack support 14 using any suitable coupling method or device which may enable the rack 21 to preferably pivot or rotate relative the rack support 14 and frame 11. In preferred embodiments, a rack 21 may be movable coupled to a rack support 14 via an axle 15 and bearing assembly 16. Optionally, a motivator 31 may comprise an axle 15 and bearing assembly 16, and the rack 21 may be movable coupled to a rack support 14 by the rack 21 being coupled to the motivator 31 which is in turn coupled to the rack support 14. A bearing assembly 16 may comprise a bearing which is a machine element that constrains relative motion to only the desired motion and reduces friction between moving parts. Preferably, the bearing assembly 16 may provide for free rotation around a fixed axis which may be provided by an axle 15 preferably configured as a cylindrical shaft.

In some embodiments, a bearing assembly 16 may comprise rotary bearings that may hold rotating components such as shafts or axles 15 within mechanical systems and transfer axial and radial loads from the source of the load to the structure supporting it. The simplest form of bearing, the plain bearing, consists of a shaft rotating in a hole. In some embodiments, a bearing assembly 16 may comprise a ball bearing and roller bearing which may include rolling elements such as rollers or balls with a circular cross-section are located between the races or journals of the bearing assembly.

Each rack 21 may comprise one or more rails 22A, 22B. Preferably, each rack 21 may comprise one or more sets of rails 22A, 22B. A set of rails 22A, 22B, may comprise two rails 22A, 22B, each having a channel 23, and the channels 23 of the two rails 22A, 22B, may face each other so that a portion of an object, such as a bioreactor 200 may be positioned in both channels so that the portion of the object may contact and rest on or be supported by the two rails 22A, 22B.

In some embodiments, each rail 22A, 22B, may comprise one or more channels 23, and each channel 23 may be formed by an upper retaining wall 24 and an opposing lower retaining wall 25 that may both be coupled to a central wall 26. Generally, an upper retaining wall 24 and a lower retaining wall 25 may be coupled to and separated by a central wall 26. The height of the central wall 26 preferably may be slightly larger than the height of a vessel coupler 203 of a bioreactor 200 so that the retaining walls 24, 25, may fit closely or snugly on opposing sides (e.g., top and bottom sides) of the vessel coupler 203 so as to minimize free play or movement of the portion of the vessel coupler 203 received in the channel 23 while allowing the portion of the vessel coupler 203 in the channel 23 to slidably move within the channel 23.

In some embodiments, a rail 22A, 22B, may comprise one or more, such as each, of an upper retaining wall 24 that may be elongated in shape, a lower retaining wall 25 that may be elongated in shape, and a central wall 25 that may be elongated in shape. In preferred embodiments, the retaining walls 24, 25, and central wall 26 may be coupled together to form a channel 23 having an elongated C-shape (elongated so that the distance between the proximal end 41 and distal end 42 of the channel 23 is the longest dimension of the rail 22). In other embodiments, retaining walls 24, 25, and central wall 26 may be coupled together to form a channel 23 of any other shape and size which may be able to receive opposing sides (e.g., top and bottom sides) of a vessel coupler 203. In further embodiments, a rack 21 and/or rail 22A, 22B, may comprise any other configuration or shape which may enable the rack 21 and/or rail 22A, 22B, to hold any number of bioreactors 200 and enable them to move in a movement circuit 70.

In some embodiments, an upper retaining wall 24 may comprise an upper retaining surface 44, a lower retaining wall 25 may comprise a lower retaining surface 45, and a central wall 26 may comprise a central surface 46, and the upper retaining surface 44, lower retaining surface 45, and central surface 46 may form the surfaces of a channel 23. In preferred embodiments, an upper retaining surface 44, a lower retaining surface 45, and a central surface 46 may be elongated and may be coupled together to form a channel 23 having an elongated C-shape. In other embodiments, an upper retaining surface 44, a lower retaining surface 45, and a central surface 46 may be coupled together to form a channel 23 of any other shape and size which may be able to receive opposing sides (e.g., top and bottom sides) of a vessel coupler 203.

As perhaps best shown in FIGS. 1 and 7, a rail 22A, 22B, may comprise one or more channels 23. Optionally, rails 22A that may be coupled to rack supports 14 and/or a motivator 31 may have one channel 23 while rails 22B that are centrally located in an array of rails 22A, 22B, may have two opposingly positioned channels 23 (channels 23 facing away or in the opposite direction of each other). Optionally, a rail 22A, 22B, may comprise a wide central wall 26 having two or more upper retaining walls 24 and two or more lower retaining walls 25 so that the rail 22A, 22B, may comprise two or more channels 23 that may face in the same direction so that the rail 22A, 22B, may support two or more horizontal rows of bioreactors 200 and two or more vertical columns of bioreactors 200.

Generally, a bioreactor 200 may be removably coupled to a rack 21 by having its vessel coupler 203 held between two adjacent rails 22A, 22B, that have their channels 23 facing each other so that opposing portions of the vessel coupler 203 are received in the two facing and adjacent channels 23. For example, a vessel coupler 203 may be held between two adjacent rails 22A, 22B, that have their channels 23 facing each other so that the vessel coupler 203, and therefore the bioreactor 200 may rest on the first upper retaining wall 24 and/or the first lower retaining wall 25 (depending on the orientation of the rail in a movement circuit 70) of the first channel 23 of the first rail 22A, 22B, and so that the vessel coupler 203, and therefore the bioreactor 200 may rest on the second upper retaining wall 24 and/or the second lower retaining wall 25 (depending on the orientation of the rail in a movement circuit 70) of the second channel 23 of the second rail 22. In this manner, a set of two rails 22A, 22B, with each rail 22A, 22B, having its channel 23 facing the other rail 22, may be configured to support one or more bioreactors 200 by receiving opposing portions of the vessel coupler 203 of each bioreactor 200 in the two channels 23 so that the vessel coupler 203 may rest on the upper retaining surfaces 44 of the retaining walls 24 and/or the lower retaining surfaces 45 of the lower retaining walls 25 (depending on the orientation of the rail in a movement circuit 70).

In preferred embodiments, the central walls 26 of two rails 22A, 22B, having adjacent channels 23 may be substantially parallel (plus or minus five degrees) to each other. For example, a first central wall 26 and its central surface(s) 46 of a first rail 22A, 22B, may be elongated in shape and a second central wall 26 and its central surface(s) 46 of an adjacent second rail 22A, 22B, the channels 23 of the rails 22A, 22B, facing each other, may be elongated in shape, and the first central wall 26 may be substantially parallel to the second central wall 26. In preferred embodiments, the upper retaining walls 24 of two rails 22A, 22B, having adjacent channels 23 may be substantially parallel (plus or minus five degrees) to each other. For example, a first upper retaining wall 24 and its upper retaining surface(s) 44 of a first rail 22A, 22B, may be elongated in shape and a second upper retaining wall 24 and its upper retaining surface(s) 44 of an adjacent second rail 22A, 22B, the channels 23 of the rails 22A, 22B, facing each other, may be elongated in shape, and the first upper retaining wall 24 may be substantially parallel to the second upper retaining wall 24. In preferred embodiments, the lower retaining walls 25 of two rails 22A, 22B, having adjacent channels 23 may be substantially parallel (plus or minus five degrees) to each other. For example, a first lower retaining wall 25 and its lower retaining surface(s) 45 of a first rail 22A, 22B, may be elongated in shape and a second lower retaining wall 25 and its lower retaining surface(s) 45 of an adjacent second rail 22A, 22B, the channels 23 of the rails 22A, 22B, facing each other, may be elongated in shape, and the first lower retaining wall 25 may be substantially parallel to the second lower retaining wall 25.

In some embodiments, the device 100 may comprise one or more arrestors 27 which may be configured to stop or govern the ability of a bioreactor 200 to enter and exit a channel 23 through a proximal end 41 so that the vessel coupler 203 may only enter and exit the channels 23 facing each other of two adjacent rails 22A, 22B, through the distal ends 42 of the channels 23. In preferred embodiments, an arrestor 27 may comprise a plate, bar, rail, or other structure that may be coupled to one or more elements of one or more racks 21 so that the arrestor 27 is coupled to or positioned proximate to the proximal end 41 of one or more channels 23. Optionally, an arrestor 27 may be coupled to one or more rails 22A, 22B, of a rack 21. Preferably, an arrestor 27 may be coupled to each rail 22A, 22B, of a rack 21, and the arrestor 27 may block the proximal end 41 of each rail's 22A, 22B, channel 23 so that a bioreactor 200 is prevented from entering and exiting that side of each rail's 22A, 22B, channel 23. In further embodiments, an arrestor 27 may comprise a protrusion, structure, fastener, or device which may be removably coupled or generally non-removably coupled to one or more rails 22A, 22B, or other element of the device 100 and which may be configured to stop or govern the ability of a bioreactor 200 to enter and exit one or both ends of a channel 23.

In some embodiments, a rack 21 may be manually rotated by a user. Optionally, the device 100 may comprise an arm, lever, wheel, etc., extending out from the pivoting axle 15, bearing assembly 16, rack 21, or other element which may be manipulated by a user to enable or to facilitate movement of a rack 21. In further embodiments, the device 100 may comprise a motivator 31 which may be configured to motivate the one or more racks 21 of the device 100 in a movement circuit 70. In some embodiments, a motivator 31 may operate an arrangement of gears which may motivate the one or more racks 21 of the device 100 in a movement circuit 70. In further embodiments, the movement directions of a motivator 31 may be controlled by a control unit 50 motivate the one or more racks 21 of the device 100 in a movement circuit 70.

In some embodiments, a rack 21 may be configured to move in a movement circuit 70 that enables the rack 21 to be completely rotated, e.g., at least a complete 360 degree revolution. For example, a user may manually and freely move a rack 21 to complete any number of full 360 degree revolutions or partial revolutions. As another example, a motivator 31 may move a rack 21 to complete any number of full 360 degree revolutions or partial revolutions.

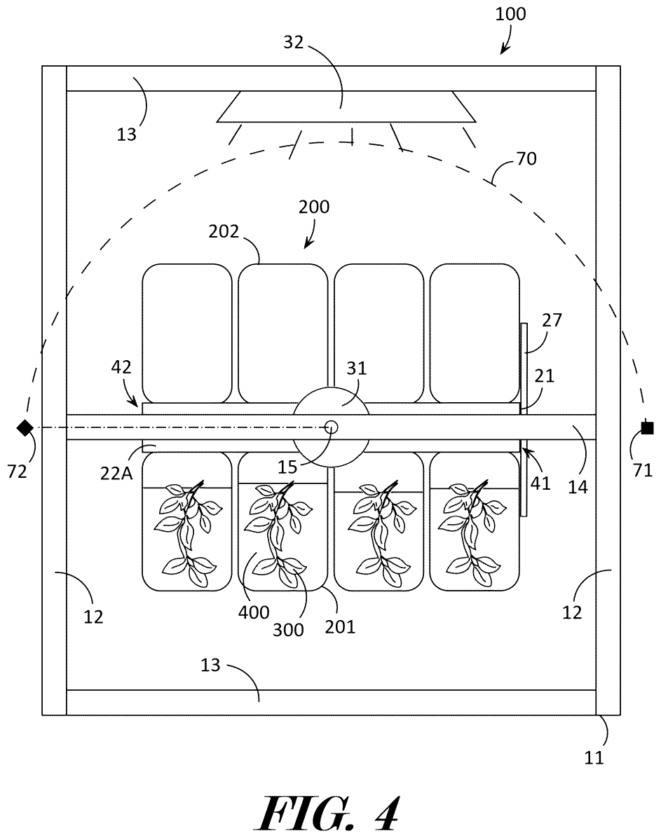

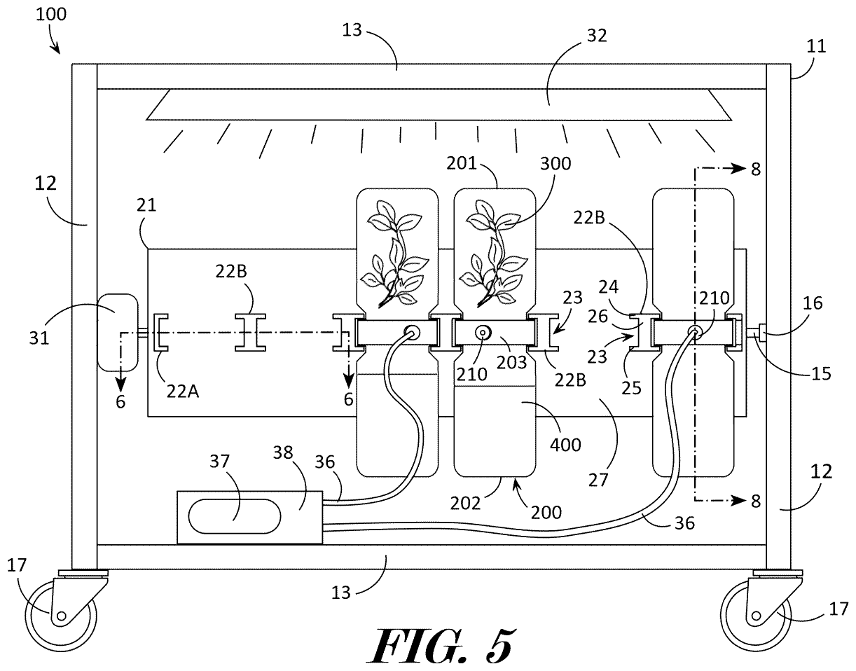

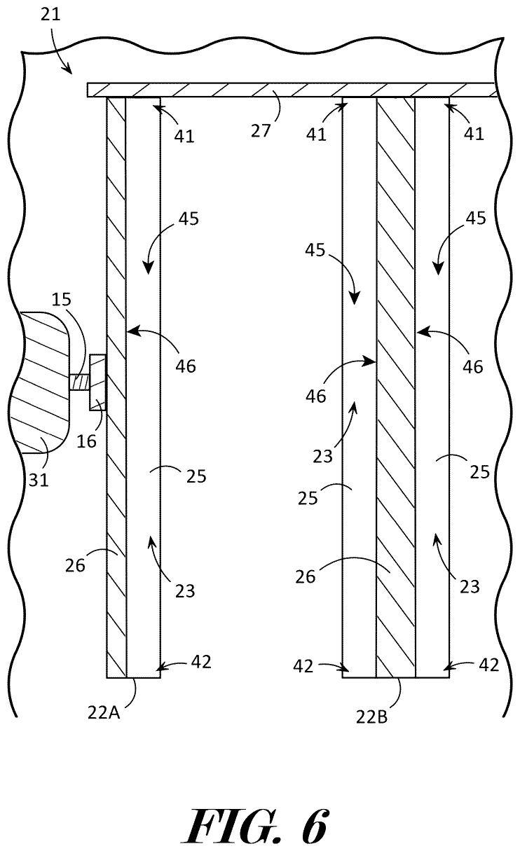

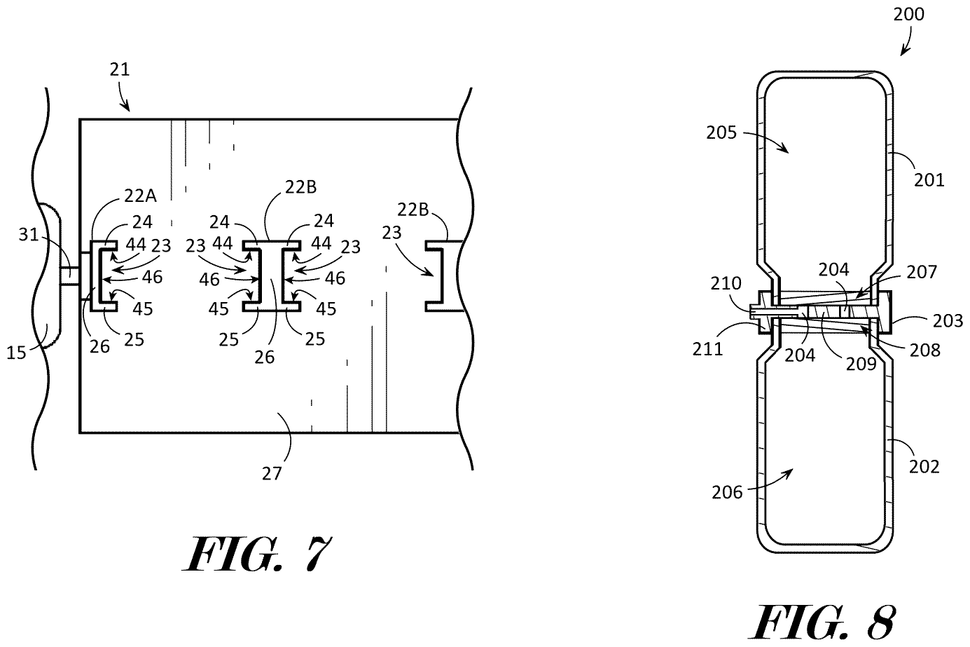

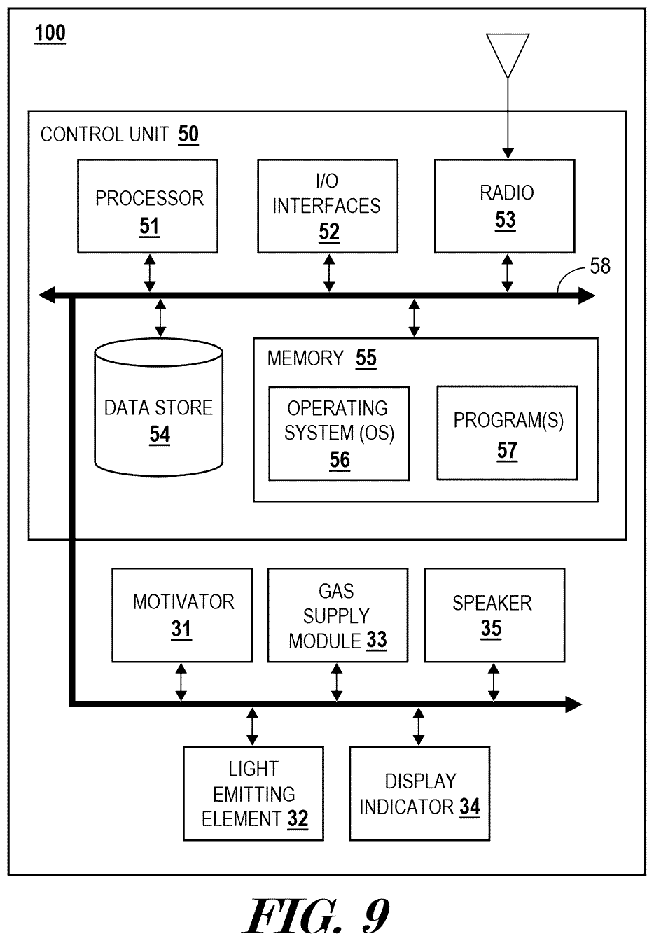

In some embodiments, a motivator 31 may be configured to move a rack 21 in a movement circuit 70 that comprises a starting and ending point 71 and a midpoint 72, and the motivator 31 may be configured to move the rack 21, and its rails 22A, 22B, and channels 23, in the movement circuit 70 in which the distal ends 42 of the channels 23 are closest to the starting and ending point 71 (FIG. 2) and in which the distal ends 42 of the channels 23 are closest to the midpoint 72 (FIG. 4). In some embodiments, the device 100 may include a motivator 31 that may be configured to move a rack 21, and its rails 22A, 22B, and channels 23, in a movement circuit 70 that comprises a starting and ending point 71 and a midpoint 72, and the device 100 may include an axle 15 that movably couples the rack 21 to the frame 11 and motivator 31 in which the axle 15 may be substantially equidistant (plus or minus five percent of the distance between the starting and ending point 71 and a midpoint 72) to both the starting and ending point 71 and the midpoint 72.

In preferred embodiments, a movement circuit 70 may comprise an approximately 180-degree arc that a rack 21 may be moved or oscillated in an axis provided by one or more axles 15 and bearing assemblies 16, optionally and a motivator 31. For example, and as shown in FIG. 2, when the rack 21 is not being fully rotated, a motivator 31 may be configured to motivate a rack 21 in a movement circuit 70 beginning with a starting and ending point 71, in which the bioreactors 200 held by the rack 21 are oriented with their first vessels 201, having plants 300, directly above their second vessels 202, having liquid exudates 400, to a midpoint 72, in which the bioreactors 200 are oriented with their second vessels 202 directly above their first vessels 201, and then the motivator 31 may be configured to motivate the rack 21 back to the starting and ending point 71 so that the distal ends 42 are only moved between 170 and 180 degrees around the axle 15. In this example, the device 100 may comprise an arrestor 27 which may prevent the bioreactors 200 from sliding out of the channels 23 when the rack 21 is between the starting and ending point 71 and midpoint 72 while allowing a user to easily slide the bioreactors into an out of the channels 23 from the end of the channels 23 that is distal to the arrestor 27. In alternative embodiments, a motivator 31 may be configured to motivate the one or more racks 21 in any other movement circuit 70, such as a movement circuit 70 having a starting and ending point 71 and midpoint 72 that are less than 180 degrees apart. Preferably, when and if operated by hand, a user will be able to completely revolve a rack 21 in any desired orientation.

In preferred embodiments, a motivator 31 may comprise a linear actuator motor. By positioning one linear actuator motor motivator 31 on one end or on both ends of a frame 11, the extending and retraction motion of a linear actuator motor motivator 31 easily moves the rack(s) that it is operably coupled to in an approximately 180-degree arc. Any type of linear actuator motor motivator 31 may be used by the device 100, including electric, pneumatic, and hydraulic cylinders. In some embodiments, a motivator 31 may comprise a brushed DC motor, brushless DC motor, switched reluctance motor, universal motor, AC polyphase squirrel-cage or wound-rotor induction motor, AC SCIM split-phase capacitor-start motor, AC SCIM split-phase capacitor-run motor, AC SCIM split-phase auxiliary start winding motor, AC induction shaded-pole motor, wound-rotor synchronous motor, hysteresis motor, synchronous reluctance motor, pancake or axial rotor motor, stepper motor, or any other type of motor. In further embodiments, a motivator 31 may comprise a hydraulic motor such as a Gear and vane motor, Gerotor motor, Axial plunger motors, Radial piston motors, or any other hydraulically motivated motor. In still further embodiments, a motivator 31 may comprise a pneumatic motor, such as a linear pneumatic motor and a pneumatic rotary vane motor.

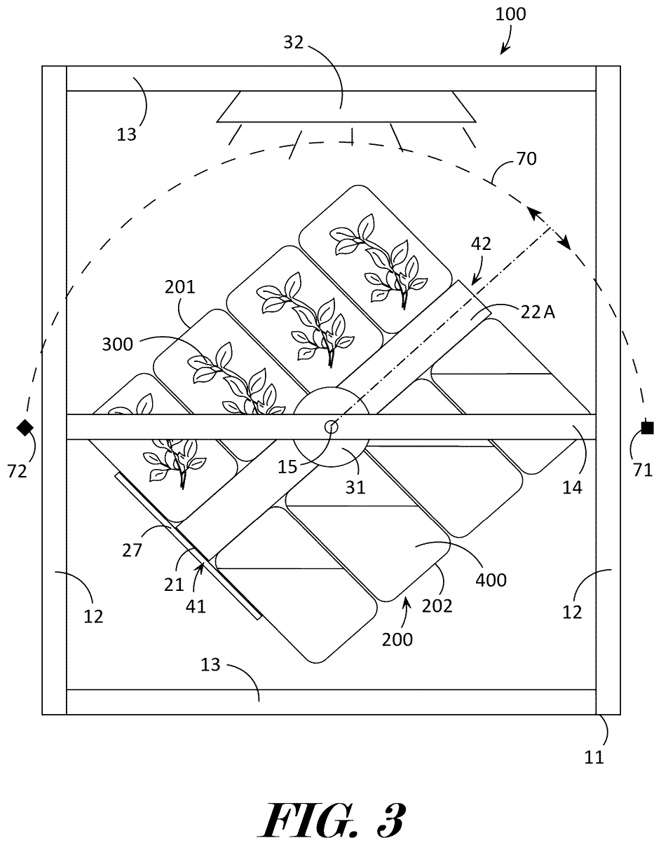

In some embodiments, the device 100 may comprise a control unit 50 which may be in electronic communication with the motor motivator 31, and the control unit 50 may be configured to control the speed, direction, and/or ability of motivator 31 move the one or more racks 21 in one or more movement circuits 70. In some embodiments and in the present example, the device 100 can be a digital device that, in terms of hardware architecture, may include a control unit 50 which may include one or more of a processor 51, input/output (I/O) interfaces 52, a radio module 53, a data store 54, and memory 55. It should be appreciated by those of ordinary skill in the art that FIG. 3 depicts the device 100 in an oversimplified manner, and a practical embodiment may include additional components or elements and suitably configured processing logic to support known or conventional operating features that are not described in detail herein.

The control unit 50 components and elements (51, 52, 53, 54, 55) are communicatively coupled via a local interface 58. The local interface 58 can be, for example but not limited to, one or more buses, circuit boards, wiring harnesses, or other wired connections or wireless connections, as is known in the art. The local interface 58 can have additional elements, which are omitted for simplicity, such as controllers, buffers (caches), drivers, repeaters, and receivers, among many others, to enable communications. Further, the local interface 58 may include address, control, and/or data connections to enable appropriate communications among the aforementioned components.

The processor 51 is a hardware device for executing software instructions. The processor 51 can be any custom made or commercially available processor, a central processing unit (CPU), an auxiliary processor among several processors associated with the control unit 50, a semiconductor-based microprocessor (in the form of a microchip or chip set), or generally any device for executing software instructions. When the processing unit 50 is in operation, the processor 51 is configured to execute software stored within the memory 55, to communicate data to and from the memory 55, and to generally control operations of the device 100 pursuant to the software instructions and/or from instructions received from a I/O interface 52. In an exemplary embodiment, the processor 51 may include a mobile optimized processor, such as optimized for power consumption and mobile applications.

The I/O interfaces 52 can be used by a user to provide user input and display system output data, such as operational status, from the system 100. The I/O interfaces 52 can include, for example, buttons, knobs, switches, LED indicator lights, LED display, LCD display, a serial port, a parallel port, a small computer system interface (SCSI), an infrared (IR) interface, a radio frequency (RF) interface, a universal serial bus (USB) interface, and the like. In some embodiments, I/O interfaces 52 may comprise buttons, knobs, switches, etc., that may be manipulated by a user to enable the user to select one or more movement circuit 70 parameters.

An optional radio module 53 or network interface enables wireless and/or wired communication to an external access device or network. A radio module 53 may enable a user to provide user input to the system 100 and to receive system 100 status data via an electronic device, such as a smartphone, tablet computer, laptop computer, desktop computer, etc., to enable the user to select one or more movement circuit 70 parameters.

The memory 55 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 55 may incorporate electronic, magnetic, optical, and/or other types of storage media. Note that the memory 55 may have a distributed architecture, where various components are situated remotely from one another, but can be accessed by the processor 51. The software in memory 55 can include one or more software programs, each of which includes an ordered listing of executable instructions for implementing logical functions. In the example of FIG. 3, the software in the memory system 55 includes a suitable operating system (O/S) 56 and program(s) 57. The operating system 56 essentially controls the execution of input/output interface 52 and other element functions, and provides scheduling, input-output control, file and data management, memory management, and communication control and related services. The operating system 56 may be, for example, LINUX (or another UNIX variant), Android (available from Google), Symbian OS, Microsoft Windows CE, Microsoft Windows 7 Mobile, iOS (available from Apple, Inc.), webOS (available from Hewlett Packard), Blackberry OS (Available from Research in Motion), and the like. The programs 57 may include various applications, add-ons, etc. configured to provide end user functionality of the device 100. In a typical example, one or more of the programs 57 may comprise instructions for controlling the functions of the motivator 31. For example, a program 57 may be used to control: the amount of time between the completion of movement circuits 70; the number of movement circuits 70 to be performed; how long the movement circuits 70 are to be performed for; the locations of a starting and ending point 71 and midpoint 72 of a movement circuit 70, etc. In further embodiments, a program 57 may be configured to operate a speaker type of I/O interface 52 to output an audible warning signal to indicate the device 100 is about to operate as a warning for the user to stop loading/unloading and to remove their hands before rotation of a rack 21 via a motivator 31 actually starts.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The control unit 50 may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by the processor 51. In addition, the main memory may be used for storing temporary variables or other intermediate information during the execution of instructions by the processor 51. The control unit 50 may further include a read only memory (ROM) or other static storage device (e.g., programmable ROM (PROM), erasable PROM (EPROM), and electrically erasable PROM (EEPROM)) coupled to the bus for storing static information and instructions for the processor 51.

In some embodiments, the device 100 may comprise one or more light emitting elements 32 which may be configured to generate light of one or more wavelengths and intensities. For example, a light emitting element 32 may comprise a light emitting diode (LED), an incandescent bulb, a fluorescent bulb, etc., which may be configured to output light that plants predominantly use for photosynthesis (such as Photosynthetically Active Radiation (PAR) 400-700 nanometers (nm) in wavelength) or for Photomorphogenesis which occurs in a wider range from approximately 260-780 nm in wavelength and includes UV and far-red radiation. Optionally, the light emitting elements 32 may be controlled and operated by a control unit 50.

In some embodiments, the device 100 may comprise a gas supply module 33 which may be used to inject a suitable gas mixture into one or more of the bioreactors 200 which may be supported by the one or more racks 21 of the device 100. Generally, a gas supply module 33 may include tubing 36 and gas regulating hardware 38 that may be used to inject a suitable gas mixture into one or more of the bioreactors 200 while still allowing the bioreactors 200 to be motivated through movement circuits 70. Tubing 36 may comprise any suitable gas conducting conduit, such as Polyvinyl Chloride (PVC) tubing, vinyl tubing, braided metal gas line, rubber gas hose, etc. Gas regulating hardware 38 may comprise a pump, such as, and/or a gas regulator which is a valve system that controls the pressure of gas for a variety of applications. Typically, regulators work by using gas to put pressure on the diaphragm, which then moves upward as controlled by a set spring. This allows a specific flow of gas to pass from the source to the device or appliance. The rate of flow and pressure can be adjusted by adjusting the control knob. Optionally, a gas supply module 33 may comprise or may be in communication with a tank of gas, a gas generator, such as a carbon dioxide generator, or other source of gas which may be useful or beneficial to plants. As an example, a gas supply module 33 may be configured to inject carbon dioxide gas into the bioreactors 200, via a port 210 in their vessel couplers 203, to enable increased growth of the propagules and eliminate or reduce the necessity of sucrose as a carbon source. In this manner, the device 100 is suitable and adaptable for Liquid Temporary Immersion Photoautotrophic (sucrose-free) Micropropagation.

In some embodiments, the device 100 may comprise one or more display indicators 34 which may be configured to visually output or display information to a user. For example, a display indicator 34 may be configured to light up to visually indicate or warn if the device 100 is powered on, if the device 100 is about to move one or more racks 21, etc. In some embodiments, a display indicator 34 may comprise a light emitting device such as a light-emitting diode (LED), incandescent bulb, etc. In still further embodiments, a display indicator 34 may comprise a Liquid crystal display (LCD), Light-emitting diode display (LED), Electroluminescent display (ELD), Electronic paper, E Ink, Plasma display panel (PDP), Cathode ray tube display (CRT), High-Performance Addressing display (HPA), Thin-film transistor display (TFT), Organic light-emitting diode display (OLED), Surface-conduction electron-emitter display (SED), Laser TV, Carbon nanotubes, Quantum dot display, Interferometric modulator display (IMOD), and/or any other device or method which may be configured to provide visual information to a user and optionally to receive input from a user, such as a touch screen display.

In some embodiments, the device 100 may comprise a speaker 35 which may be used to produce a plurality of sounds at a plurality of volume levels. Preferably, a speaker 35 may be configured to produce sounds which may be used to audibly appraise or warn a user of the status of one or more elements of the device 100 and/or of one or more conditions that the device 100 is in. For example, if a rack 21 is about to be moved or is being moved, the control unit 50 may cause the speaker 35 to emit a siren or other warning sound. A speaker 35 may comprise a buzzer, a piezoelectric sound producing device, a dielectric elastomer sound producing device, a moving coil loudspeaker, an electrostatic loudspeaker, an isodynamic loudspeaker, a piezo-electric loudspeaker, or any other device capable of producing one or more sounds.

While some exemplary shapes and sizes have been provided for elements of the device 100, it should be understood to one of ordinary skill in the art that the frame 11, one or more racks 21, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 100 may be made from or may comprise durable materials such as aluminum, steel, other metals and metal alloys, wood, hard rubbers, hard plastics, fiber reinforced plastics, carbon fiber, fiberglass, resins, polymers or any other suitable materials including combinations of materials. Additionally, one or more elements may be made from or may comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A bioreactor liquid agitation device, the device comprising:
a frame;
a rack movably coupled to the frame, wherein the rack comprises a first rail, a second rail, and an arrestor, wherein the first rail comprises a first channel formed by a first upper retaining wall and a first lower retaining wall that are each coupled to a first central wall, wherein the first channel comprises a first distal end and a first proximal end, wherein the second rail comprises a second channel formed by a second upper retaining wall and a second lower retaining wall that are each coupled to a second central wall, wherein the second channel comprises a second distal end and a second proximal end, and wherein the first channel faces the second channel, wherein the arrestor is coupled to the first rail so that the first proximal end forms a portion of the first channel that is closest to the arrestor; and
a motivator which is configured to move the rack relative to the frame.

2. The device of claim 1, wherein the motivator is configured to move the rack in a movement circuit that comprises a starting and ending point and a midpoint, wherein the motivator is configured to move the rack in the movement circuit in which the first distal end and the second distal end are closest to the starting and ending point and in which the first distal end and the second distal end are closest to the midpoint.

3. The device of claim 1, wherein the first central wall is elongated in shape and the second central wall is elongated in shape, and wherein the first central wall is substantially parallel to the second central wall.

4. The device of claim 1, wherein the second rail is coupled to the arrestor.

5. The device of claim 1, wherein the second rail comprises a third channel.

6. The device of claim 1, wherein the first rail is movably coupled to the frame via an axle and a bearing assembly.

7. The device of claim 6, wherein the motivator is configured to move the rack in a movement circuit that comprises a starting and ending point and a midpoint, and wherein the axle is substantially equidistant to both the starting and ending point and the midpoint.

8. The device of claim 7, wherein the motivator is configured to move the rack in the movement circuit in which the first distal end and the second distal end are positioned closest to the starting and ending point, in which the first distal end and the second distal end positioned closest to the midpoint, and in which the first distal end and the second distal end are returned to be positioned closest to the starting and ending point, and wherein the first distal end and the second distal end are only moved between 170 and 180 degrees around the axle.

9. The device of claim 1, further comprising a light emitting element.

10. The device of claim 9, wherein the light emitting element emits light between 260-780 nm in wavelength.

11. A bioreactor liquid agitation device for use with one or more bioreactors, each bioreactor having a first vessel and a second vessel which are coupled together via a vessel coupler and the vessel coupler providing fluid communication between the first vessel and the second vessel, the device comprising:
a frame;
a rack movably coupled to the frame, wherein the rack comprises a first rail, a second rail, and an arrestor, wherein the first rail comprises a first channel formed by a first upper retaining wall and a first lower retaining wall that are each coupled to a first central wall, wherein the first channel comprises a first distal end and a first proximal end, wherein the second rail comprises a second channel formed by a second upper retaining wall and a second lower retaining wall that are each coupled to a second central wall, wherein the second channel comprises a second distal end and a second proximal end, and wherein the first channel faces the second channel, wherein the arrestor is coupled to the first rail so that the first proximal end forms a portion of the first channel that is closest to the arrestor, wherein the bioreactor is removably coupled to the rack by positioning the vessel coupler in the first channel through the first distal end and in the second channel through the second distal end, and wherein the arrestor prevents the vessel coupler from exiting the first channel through the first proximal end; and
a motivator which is configured to move the rack relative to the frame.

12. The device of claim 11, wherein the motivator is configured to move the rack in a movement circuit that comprises a starting and ending point and a midpoint, wherein the motivator is configured to move the rack in the movement circuit in which the first distal end and the second distal end are closest to the starting and ending point and in which the first distal end and the second distal end are closest to the midpoint.

13. The device of claim 11, wherein the first central wall is elongated in shape and the second central wall is elongated in shape, and wherein the first central wall is substantially parallel to the second central wall.

14. The device of claim 11, wherein the second rail is coupled to the arrestor.

15. The device of claim 11, wherein the second rail comprises a third channel.

16. The device of claim 11, wherein the first rail is movably coupled to the frame via an axle and a bearing assembly.

17. The device of claim 16, wherein the movement circuit comprises a starting and ending point and a midpoint, and wherein the axle is substantially equidistant to both the starting and ending point and the midpoint.

18. The device of claim 11, further comprising a light emitting element.

19. The device of claim 18, wherein the light emitting element emits light between 260-780 nm in wavelength.

20. The device of claim 11, further comprising a gas supply module that is coupled to the vessel coupler, wherein the gas supply module is configured to supply a gas into the first vessel and the second vessel via the vessel coupler.
